(19) United States
(12) Patent Application Publication
(10) Pub. No.: US 2026/0315562 A1
(43) Pub. Date: Oct. 15, 2026

(54) NOVEL FEED-ADDITIVE COMPOSITION

(71) Applicant: KYNTOX BIOTECH INDIA PRIVATE LIMITED, Bangalore (IN)

(72) Inventor: Gopi Kadiyala, Bengaluru (IN)

(73) Assignee: KYNTOX BIOTECH INDIA PRIVATE LIMITED, Bangalore (IN)

(21) Appl. No.: 19/660,138

(22) Filed: Apr. 27, 2026

Related U.S. Application Data
(63) Continuation of application No. PCT/IN2024/052125, filed on Oct. 24, 2024.

(30) Foreign Application Priority Data
Oct. 26, 2023 (IN) 202341072903

(51) Int. Cl. A61K 36/53; A61K 9/00; A61K 31/23; A61K 31/4525; A61K 31/717; A61K 31/734; A61K 33/10; A61K 38/16; A61P 31/04

(52) U.S. Cl. CPC A61K 36/53; A61K 9/0053; A61K 31/23; A61K 31/4525; A61K 31/717; A61K 31/734; A61K 33/10; A61K 38/164; A61P 31/04

(57) ABSTRACT

The present invention relates to a novel feed-additive composition for use as prophylactic and therapeutic agents for the treatment of fungal, viral and bacterial infections in humans, animals and aquatic animals, including white spot shrimp virus (WSSV) infection in aquatic species, including crab and shrimp. The additive composition comprises glycerol monolaurate, rosemary oil, nisin peptide, calcium carbonate, piperin, sodium alginate, and carboxymethyl cellulose, and additive composition comprises glycerol monolaurate and saponin-rich extract in the ratio disclosed hereinabove. The said additive composition in a lower concentration can be used for prevention and in a higher concentration can be used for treatment of WSSV infection in aquaculture species. The present invention also relates to a process of manufacturing of the additive composition such as hereinbefore described.

### FIELD OF INVENTION

The present invention relates to a novel, additive composition for improving disease resistance against viral and bacterial infections. More particularly, the present invention relates to an additive composition for improving disease resistance against viral and bacterial infections in human, animal, plants, and aquatic animals, including crab, fish and shrimp, preferably used as an aqua-culture feed additive. The present invention further relates a process of manufacturing the novel feed-additive composition.

### BACKGROUND OF THE INVENTION

The subject matter discussed in the background section should not be assumed to be prior art merely because of its mention in the background section. Similarly, a problem mentioned in the background section or associated with the subject matter of the background section should not be assumed to have been previously recognized in the prior art. The subject matter in the background section merely represents different approaches, which in and of themselves may correspond to implementations of the claimed invention.

Aquatic species play a crucial role in providing humans with vital nutrients, including but not limited to omega-3 fatty acids, iodine, vitamins B2 and D. Presently, aquaculture accounts for about half of the world's supply of fish and other aquatic species. Approximately 90% of the global aquaculture production is concentrated in the Asia-Pacific region, with the top producers being China, India and Indonesia. Fish farming in developing countries provides domestic food security and supports rural livelihoods with the production of high value fish destined for export. From 1995-2020, the world employment for fishers and aquaculture farmers has increased two to three times. The aquaculture accounts for 57.3 percent of 214 million tons of total aquatic production which is divided between aquatic animals and algae. Moreover, globally, the fish and aquatic species provide about 17% of animal protein eaten by people, with a few countries in Asia and Africa even reaching up to 50%. (FAO. 2022, The State of World Fisheries and Aquaculture 2022. Towards Blue Transformation. Rome, FAO.)

In recent decades, the aquaculture business has expanded dramatically in response to the increasing need for food worldwide, greatly boosting the economies of the nations that engage in this activity. Global aquaculture market is estimated to be USD 289.6 Billion in 2022 and expected to reach USD 421 Billion by 2023 at a growth rate of 5.5% (Aquaculture market industry report, 2022-23).

It is well known that infectious diseases are by far the greatest limiting factor to expanding aquaculture production. In the recent years, emerging infectious diseases have caused a significant production loss in many species across the world.

The aquaculture operations in the tropics experience higher cumulative mortalities and faster progression of diseases and this could be exacerbated by climate change leading to selection of virulent pathogens that have the potential to spread. This can result in introduction and spread of more virulent pathogens to natural fisheries and aquaculture landscapes, threatening a significant part of the global supply of nutritious animal foods (UNFCC report on Climate Change and Aquatic Animal Disease by Chadag Mohan, World Fish Center, Penang, Malaysia). More recently, Asian sea bass ‘Lates calcarifer’, also known as ‘barramundi’ were infected by the scale drop disease virus (SDDV) leading to a huge economic and production loss in Singapore.

Shrimps, are one of the most reared aquatic animals. However, intensive farming may increase the possibilities of the entrance of emergent pathogens, which may cause severe economic losses and reduce the profitability of aquaculture, across the globe. The disease severity and economic losses depend upon the type of harmful virus and bacteria that cause the deadly disease in aquaculture animals, especially shrimp and fish. Various pathogenic viruses and bacteria, more precisely White Spot Syndrome Virus (WSSV) which causes the White Spot Disease and some of the genera of gram-negative bacteria, particularly Vibrio species which causes Vibriosis are known to contribute to huge economic losses in aquaculture.

Amongst the shrimps in aquaculture, Litopenaeus vannamei is one of the most widely farmed shrimp species in the world and is valued for its rich nutritional value. The shrimp-culture business has suffered significant economic losses worldwide, due to various viral and bacterial infections affecting the growth and survival of the animals. The major infectious diseases include White Spot Disease (WSD) and Vibriosis caused by WSSV and Vibrio species, respectively. These diseases cause a cumulative mortality of up to 100% within 3-10 days, if untreated.

White Spot Disease (WSD) is caused by White Spot Syndrome Virus (WSSV), a rod-shaped to elliptical, highly lethal, stress-dependent DNA virus which belongs to the family Nimaviridae and genus Whispovirus. The WSSV contains a large circular double-stranded DNA (ds-DNA) genome with diameters ranging from 292.9-307.2 kb, and the virulence of the various isolates varies. This virus infects a broad variety of aquatic crustaceans, including crabs, crawfish, squilla, copepods, tiger prawns, and shrimps, among others by vertical and horizontal transmission, with varying degrees of mortality. This disease and mortality are more common in juvenile shrimps (1-5 g). The WSSV outbreaks are usually induced by rapid changes in salinity, and temperature with temperatures of 16-30 degree Celsius being conducive for outbreaks. Further, stocking in cold season is another predisposing factors of WSD outbreaks (UNFCC report on Climate Change and Aquatic Animal Disease by Chadag Mohan, World Fish Center, Penang, Malaysia). The clinical indication of WSD is the presence of white calcium deposits of 0.5-2.0 mm diameter on the cuticle of the shrimp cephalothorax or carapace in infected shrimp is a clinical evidence of the WSSV infection. These white patches can be clearly seen by removing the carapace and any connected tissue and inspecting the cuticle in natural light.

Vibriosis in shrimp is caused by gram-negative bacteria, particularly Vibrio species that can cause a variety of disorders, including shell disease, zoea II syndrome, septic hepatopancreatic necrosis, early mortality syndrome, and acute hepatopancreatic necrosis disease. Other diseases related with Vibrio infection in shrimp include red disease, “seagull syndrome”, “Bright red” syndrome, luminous vibriosis, “sea disease,” and “Bright red” syndrome. Therefore, preventing the outbreak of these diseases is critical to aquaculture.

As per the Indian Council of Agricultural Research (ICAR) report of 2016, there are no existing treatments for While Spot Disease (WSD). Previously, prevention of white spot disease has been attempted using probiotics, vaccines, immunostimulants, and environmental quality management, however the shrimp mortality in ponds and hatcheries due to diseases continue to occur (Muliani et al., 2021 IOP Conf Ser.: Earth Environ. Sci. 860 012049).

In the past, antibiotics were widely used in aquaculture. However, intensive farming leads to the unencumbered use of antibiotics, which results in residues of such antibiotics in the aquatic products. Such residues end up causing development of antibiotic resistance in aquatic animals. There are several studies that evidences the transmission of antibiotic resistance between aquatic species to terrestrial animals. This poses a huge threat to the human and animal health world-wide. As a result, this has caused the Food and Drug Administration (FDA) to prohibit the use of several antibiotics in aquaculture used in the treatment of various viral and bacterial infections.

Therefore, there is a need for providing an effective product that is capable of effectively treating and/or preventing viral and bacterial diseases in aquatic species, including but not limited to shrimps and fish. Moreover, the product required for the said purpose must comprise substances identified as Generally Regarded as Safe (GRAS) by the FDA to ensure safety profile of the product.

Some efforts have been made in the past to meet this requirement by providing biological compositions comprising dietary supplementation of glycerol monolaurate (GML) to treat crayfish (Procambarus clarkii) infected with white spot syndrome virus (WSSV). The GML treatment was observed to reduce the mortality of WSSV-infected crayfish and reduced the WSSV copy number (Qian et. al., 2021 and Gong et.al 2022). However, there remains a need for an improved product for an effective treatment in addition to a prophylactic treatment of WSD in aquatic animals.

In the present invention, a novel biological composition is provided that can be added to aquaculture feed to effectively reduce and/or prevent the incidences of infectious diseases such as White Spot Disease (WSD) and Vibriosis caused by WSSV and Vibrio species, respectively, in aquatic animals, including but not limited to shrimp and fish.

### SUMMARY OF THE INVENTION

The instant disclosure provides a novel aquaculture feed-additive composition. This summary is not intended to identify essential features of the claimed subject matter, nor is it intended for use in determining or limiting the scope of the claimed subject matter.

The feed-additive composition of the invention in general comprises a fatty acid, an anti-microbial essential oil, a bacteriocidin, an inorganic salt of Calcium, a naturally-derived alkaloid, a salt of alginic acid, and cellulose derivatives for the effective prevention and treatment of diseases, including white spot disease caused by WSSV. These components of the composition have been commercially procured in order to produce the present composition that can be used as a feed additive or feed supplement to aquatic animals that may be susceptible to or have been infected with bacterial and/or viral infections, such as but not limited to Vibrio species and WSSV.

The present invention relates to a novel aquaculture feed-additive composition which can be added to aqua culture feed for improving disease resistance against viral and bacterial infections, particularly wherein the composition is capable of reducing and/or preventing viral and bacterial diseases in aquatic species, such as shrimps fish and crabs. The said aquaculture feed-additive composition can be used to treat viral and bacterial diseases such as white spot syndrome virus (WSSV) and Vibriosis respectively, in aquatic animals.

According to an embodiment of the present invention, provided herein is a novel aquaculture feed-additive composition comprising glycerol monolaurate (GML), Nisin, Vitamin C, Calcium carbonate, Colostrum enriched with IgG antibodies, Carboxymethyl cellulose, Sodium alginate and Piperin.

In a preferred embodiment, the novel aquaculture feed-additive composition comprises Glycerol monolaurate (GML), Rosemary oil, Nisin peptide, Calcium carbonate, Piperin, Sodium alginate and Carboxymethyl cellulose for the prevention and treatment of diseases which include but are not limited to white spot disease in shrimps.

The novel feed-additive composition disclosed herein works by cleaving the viral envelope, thereby effectively preventing viral replication and killing the viral cells. Furthermore, the composition improves the innate-immunity of the host cells.

According to another embodiment of the present disclosure, provided herein is a novel aquaculture feed-additive composition comprising Glycerol monolaurate, Rosemary oil, Nisin peptide, Calcium carbonate, Piperin, Sodium alginate, Carboxymethyl cellulose, Hyperimmuned egg powder, antibodies, such as IgY and any fragment thereof, including and not limited to single chain variable fragment (scFv), Fab, F(ab′)2 fragments, Fc fragment, among others for the prevention and treatment of diseases which include but are not limited to white spot disease in shrimps.

In a preferred embodiment of the present disclosure, provided herein is a novel aquaculture feed-additive composition comprising 5-71% w/w of glycerol monolaurate, 5-10% w/w of rosemary oil, 5-10% w/w of nisin peptide, 5-20% w/w of calcium carbonate, 2-5% w/w of piperin, 2-5% w/w of sodium alginate, and 2-5% w/w of carboxymethyl cellulose for the prevention and treatment of white spot disease in shrimps.

According to another embodiment of the present disclosure, provided herein is a novel aquaculture feed-additive composition comprising glycerol monolaurate, rosemary oil, nisin peptide, calcium carbonate, piperin, sodium alginate and carboxymethyl cellulose for the prevention of white spot syndrome virus infection by administering a dosage of 2-7 g/kg as a prophylactic dosage.

According to a yet another preferred embodiment, provided herein is a novel aquaculture feed-additive composition comprising glycerol monolaurate, rosemary oil, nisin peptide, calcium carbonate, piperin, sodium alginate and carboxymethyl cellulose for the prevention of white spot syndrome virus infection by administering a dosage of 5 g/kg as a prophylactic dosage.

According to further embodiment of the present invention, provided herein is a novel aquaculture feed-additive composition comprising glycerol monolaurate, rosemary oil, nisin peptide, calcium carbonate, piperin, sodium alginate and carboxymethyl cellulose for the treatment of white spot syndrome virus infection by administering a dosage of 7-15 g/kg as a therapeutic dosage.

According to a preferred embodiment of the present disclosure, provided herein is a novel aquaculture feed-additive composition comprising glycerol monolaurate, rosemary oil, nisin peptide, calcium carbonate, piperin, sodium alginate and carboxymethyl cellulose for the treatment of white spot syndrome virus infection by administering a dosage of 10 g/kg as a therapeutic dosage.

According to yet another embodiment of the present disclosure, provided herein is a process for preparing the novel aquaculture feed-additive composition as described herein.

Yet another embodiment of the present disclosure, provided herein is an aquaculture feed-additive composition that comprises glycerol monolaurate and saponin-rich extract. The saponin-rich extract of the said aquaculture feed-additive composition is derived from Rosmarinus officinalis L. Yet another embodiment of the present disclosure, provided herein is an additive composition that comprises glycerol monolaurate and saponin-rich extract in the treatment of bacterial and viral diseases. According to a preferred embodiment of the present disclosure, provided herein is a composition that comprises 5-71% w/w glycerol monolaurate and 2-20% w/w saponin-rich extract in the treatment of bacterial and viral diseases in human, animal and plants. According to a preferred embodiment of the present disclosure, provided herein the viral diseases include influenza viruses among others. According to a preferred embodiment of the present disclosure, provided herein the viral diseases include influenza viruses among others. According to a preferred embodiment of the present disclosure, provided herein the composition is useful in the treatment of gut and respiratory diseases in humans. According to a preferred embodiment of the present disclosure, provided herein the composition may be used alone or in combination with other pharmaceutical drugs in the treatment of diseases and maintain gut microbiome in humans and animals.

### BRIEF DESCRIPTION OF ACCOMPANYING DRAWINGS

The summary, as well as the following detailed description, are further understood when read in conjunction with the appended drawings. For the purpose of illustrating the invention, exemplary embodiments of the invention are shown in the drawings, however, the invention is not limited to the specific disclosure of the drawings

FIG. 1 provides an illustration representing a flow chart for the production of the novel feed-additive composition.

FIG. 2 provides a pictorial representation of carapace of a deceased WSSV-infected shrimp showing the white spots which is indicative of the infection.

FIG. 3 provides a pictorial representation of carapace of healthy harvested shrimp from the treatment group.

FIG. 4 provides a graphical representation of various CT values of PCR results in Pond 1 for WSSV.

FIG. 5 provides a graphical representation of weekly sampling data of pond 1 treated with the composition at a dosage of 10 g/kg (treatment group) and pond 2 without using composition at 10 g/kg (negative control group).

FIG. 6 provides a graphical representation of animal survival data with Saponins-rich extract against WSSV challenge.

FIG. 7 provides a graphical representation of animal survival data with GML against WSSV challenge.

FIG. 8 provides a graphical representation of animal survival data caused by the synergistic effect of treatment with GML and Saponins-rich extract against WSSV challenge.

### DETAILED DESCRIPTION OF THE INVENTION

This disclosure may be more fully appreciated by reference to the following description, including the following glossary of terms and the concluding examples.

The terms and words used in the following description and claims are not limited to the bibliographic meanings, but are merely used by the inventor to enable a clear and consistent understanding of the invention. Accordingly, it should be apparent to those skilled in the art that the following description of exemplary embodiments of the present invention are provided for illustrative purposes only and not for the purpose of limiting the scope of the invention as defined herein. Although any methods and materials similar or equivalent to those described herein can be used in the practice or testing of the present invention, the preferred methods and materials are described.

### 1. DEFINITIONS

As used in the description herein and throughout the claims that follow, the meaning of “a,” “an,” and “the” includes plural reference unless the context clearly dictates otherwise. In addition, as used in the description herein, the meaning of “in” includes “in” and “on” unless the context clearly dictates otherwise.

Unless indicated otherwise, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

Some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. The terms “about” or “approximately” as used herein, when referring to a numerical value or range, allow for a degree of variability in the value or range, for example, within 10% (i.e., ±10%), within 5% (i.e., ±5%), or within 2.5% (i.e., ±2.5%) of a stated value or of a stated limit of a range.

Throughout the description and claims of this specification, the words “comprise” and “contain” and variations of the words, for example “comprising” and “comprises”, mean “including but not limited to”, and are not intended to (and do not) exclude other components.

Unless otherwise indicated, each of the individual feature or embodiments of the present specification are combinable with any other individual feature or embodiment that are described herein, without limitation. Such combinations are specifically contemplated as being within the scope of the present invention, regardless of whether they are explicitly described as a combination herein.

Although the experiments have been performed on white spot symptom virus-infected shrimp, the same does not limit the scope of the invention. In fact, the same results may be expected in other aquatic animals infected with other bacterial or viral infections.

Disclosed embodiments provide a novel aquaculture feed-additive composition that comprises a fatty acid, an anti-microbial essential oil, a bacteriocidin, an inorganic salt of Calcium, a naturally-derived alkaloid, a salt of alginic acid, and cellulose derivatives.

As used herein, the expression “feed-additive” refers to supplemental ingredients added to the aquaculture feed to protect the labile nutrients, improve nutrient availability and overall efficiency. Common feed additives include synthetic amino acids, vitamins, binders, antioxidants, preservatives, prophylactic medicines (antibiotics), hormones, antimicrobial agents, and feeding stimulants, among others. Feed-additives are usually formulated with high precision to deliver balanced nutrition in the form of pellets (dry or wet) along with feed stock and added to the aquaculture for the aquatic animals to feed on them. Alternatively, additives are added to the aquaculture as a supplementary treatment.

Common aquaculture animals include, fish, starfish, seahorse, crab, molluscs, such as oysters, crustaceans, such as shrimps, crayfish, prawns and lobsters.

Most commonly reared shrimp species are P. enaeus monodon, P. indicus, Metapenaeus ensis, M monoceros, M brevicornis, Penaeus semisulcatus, P. merguiensis and L. vannamei.

As used herein, the term ‘Fatty acids’ may be selected from a fatty acid glycerides, Glyceryl Mono Laurate (GML), 1-monocaprylin (C8:0), 1-monocaprin (C10:0), 1-monolaurin (C12:0), 1-monomyristin (C14:0), 1-monopalmitin (C16:0), and 1-monostearin (C18:0) and the like.

As used herein, the term ‘Anti-microbial essential oil’ may be selected from Angelica, bergamot, lemongrass, mandarin, mint, caraway, celery, citronella, coriander, eucalyptus, geranium, petitgrain, pine, juniper, lavandin, lavander, lemon, orange, peppermint, rosemary, sage, and thyme, and the like (Chouhan S. et. al., 2017).

As used herein, the term ‘Bacteriocidins’ may be selected from nisin, epidermin, gallidermin, Pep5, and subtilin and the like (Meade E. et. al., 2020 Jan. 16; 9(1):32).

As used herein, the term ‘Inorganic salt of Calcium’ may be selected from calcium chloride, calcium carbonate and calcium phosphate and the like.

As used herein, the term ‘Naturally-derived alkaloid’ may be selected from piperine, capsaicin or gingerol; glucosinolates such as sinalbin, sulforaphen or isopropyl mustard oil; orxanthophylls, usnic acid, berberine, curcumin, delphinidin, hypericin, chrysarobin, esculetin, cinchonamine, cinnamaldehyde, myristic acid, limonene, amarogentin and safrole and the like.

As used herein, the term ‘Salt of alginic acid’ may be selected from sodium alginate, potassium alginate and magnesium alginate and the like.

As used herein, the term “essential oil” refers to the concentrated hydrophobic liquid containing volatile chemical compounds obtained from plants. They are usually obtained using double distillation method. Rosemary oil refers to the essential oil obtained from the leaves of Rosemary plant or Salvia rosmarinus.

As used herein, the term “bacteriocins” are peptide toxins produced by microorganisms which are part of the immune response that bacteria possess to inhibit other closely related bacterial strains. “Nisin” is a bacteriocin produced by some strains of the Lactococcus lactis bacterium. It belongs to a class of thermostable cationic antibacterial peptides known as antibiotics that are made up of unusual amino acid residues that form lanthionine rings.

As used herein, the term “alkaloid” refers to naturally occurring organic nitrogen-containing bases a variety of which are present in most plants. “Piperine” or “Piperin” is a phytochemical found in the fruits and roots of plants such as Piper nigrum L. and Piper longum. Chemically, piperine is an alkaloid and the structure is composed of three subunits: an amide function constituted by a piperidine ring with α-β-unsaturated carbonyl moiety, a 1, 3-benzodioxole group, also called piperonal nucleus and a butadiene chain.

As used herein, the expression “Inorganic salt of Calcium” refers to various salts containing Calcium in them which may be used for calcium supplementation in feed. The said calcium salts may also be used as a binding agent in formulations. A preferred Calcium salt disclosed herein is calcium carbonate which has a chemical formula of CaCO₃.

As used herein, the expression “salt of alginic acid” is naturally found in brown algae and has been used to refer to alginic acid salts that may be used as thickening agents in feed formulation. The salts used may be sodium or potassium alginate. A preferred component disclosed herein is sodium alginate that has a chemical formula of NaC₆H₇O₆ and is a cell-wall component of marine brown algae. It comprises of 1, 4-β-d-mannuronic (M) and α-1-guluronic (G) acids.

Cellulose derivatives have widely been used as emulsifier, stabiliser, thickener, gelling agent and binders. It is also used in food products to control crystal formation, maintain the desired texture and increase shelf life of the products. A preferred component disclosed herein is carboxymethyl cellulose.

As used herein, the term “hyperimmunized egg” refers to a whole egg obtained from an egg-producing animal maintained in a hyperimmune state, i.e. an egg-producing animal that has been hyperimmunized. The term “hyperimmunized egg powder” refers to a powder of the hyperimmunized egg or powder of any product obtained from a hyperimmunized egg. As used herein, the term “antibody fragments” refers to any fragment derived from a functional antibody, such as fragment antigen-binding region (Fab), single-chain variable fragment (scFv), F(ab′)2 fragment antibodies, and fragment crystallizable region (Fc).

As described herein, the term “white spot disease or WSD” refers to the infection caused by white spot syndrome virus (WSSV). The clinical indication of WSD as described herein is the presence of white calcium deposits of 0.5-2.0 mm diameter on the cuticle of the shrimp cephalothorax or carapace in infected shrimp is a clinical evidence of the WSSV infection. These white patches can be clearly seen by removing the carapace and any connected tissue and inspecting the cuticle in natural light.

As described herein, the term “Vibriosis” refers to the infection caused by Vibrio bacteria. Many Vibrio species are non-pathogenic and can be commonly found in healthy farmed aquatic animals. However, some Vibrio species and strains are pathogenic leading to a variety of ‘vibriosis’ diseases. However, sea-food Vibriosis poses a risk of infecting consumers with vibriosis. Most common vibrio species causing infections in aquatic species, include V. alginolyticus, V. alginolyticus, V. campbellii, V. harveyi, V. mimicus, and V. parahaemolyticus among others (Cecilia de Souza Valente et.al. 2021).

As used herein, “saponin-rich extract” are as known in the art and has been used in the ordinary meaning that a person of average skill in the art may use and understand. The “saponin-rich extract” refers to plant extracts which are rich in saponins with 2-20% w/w. Several plants may be used to prepare the “saponin-rich extract”, such as English ivy, Angelica, bergamot, lemongrass, mandarin, mint, caraway, celery, citronella, coriander, eucalyptus, geranium, petitgrain, pine, juniper, lavandin, lavander, lemon, orange, peppermint, rosemary, including Rosmarinus officinalis L, sage, and thyme. Saponins are secondary metabolites which as well-known in the art are a vital part of plant defense system from pathogens and predators.

The expressions “negative control group”, “placebo group” and “treatment group” has been used in the ordinary meaning that a person of average skill in the art may use and understand. The “negative control group” refers to aquatic animals which were fed with a normal feed without the innovative feed-additive. The expression “treatment group” refers to aquatic animals which were fed with the novel feed-additive composition described herein.

An embodiment of the present disclosure, provided herein comprises a novel, aquaculture feed-additive composition comprising a fatty acid, an anti-microbial essential oil, a bacteriocidin, an inorganic salt of Calcium, a naturally-derived alkaloid, a salt of alginic acid, and cellulose derivatives selected from the groups disclosed herein, for the prevention and treatment of bacterial and viral diseases in aquatic animals, including fish and shrimp. The present invention further relates a process of manufacturing the novel feed-additive composition.

### 2. FEED-ADDITIVE COMPOSITION

Another embodiment of the present invention, is a novel aquaculture feed-additive composition comprising glycerol monolaurate, rosemary oil, nisin peptide, calcium carbonate, piperin, sodium alginate and carboxymethyl cellulose for the prevention and treatment of diseases which include but are not limited to white spot disease in shrimps.

Glyceryl Mono Laurate (GML) is a representative fatty acid glyceride that is frequently used in food, cosmetics, and nutritional supplements as a surfactant and emulsifier. The FDA in the United States regards it as a safe natural substance with a variety of applications. GML has efficient bactericidal action against a variety of Gram-positive bacteria and is a bacteriostatic agent that is resistant to a number of diseases. Additionally, GML exhibits antiviral activity against enveloped viruses in vitro and has powerful inhibitory effects on a variety of enveloped viruses.

Rosemary oil is sometimes used in aquatic feedstock, particularly in the aquaculture industry. One of the main purposes of using rosemary oil in aquatic feed is to improve the quality and preservation of the feed, as well as to provide potential health benefits to the aquatic animals being raised. Some studies suggest that rosemary oil may have stress-reducing effects on aquatic animals.

Nisin is a bacteriocin generated by some strains of the Lactococcus lactis bacterium. It belongs to a class of thermostable cationic antibacterial peptides known as lantibiotics that are made up of unusual amino acid residues that form lanthionine rings. Nisin received the GRAS designation (generally recognised as safe) from the FDA in 1988. Nisin is utilized as a natural food preservative, notably in processed cheese, due to its broad range of antibacterial activities (Malaczewska. Et al., MC Vet Res 15, 318 (2019)).

Piperine is a phytochemical found in the fruits and roots of plants such as Piper nigrum L. and Piper longum. Chemically, piperine is an alkaloid and the structure is composed of three subunits: an amide function constituted by a piperidine ring with α-β-unsaturated carbonyl moiety, a 1, 3-benzodioxole group, also called piperonal nucleus and a butadiene chain. It has very rich pharmacological activities such as anti-bacterial, anti-viral, anti-inflammatory, immunosuppressive, anti-cancer, neuroprotective, and antioxidant effects.

In a preferred embodiment of the invention, provided herein is a novel aquaculture feed-additive composition comprising 5-71% w/w of glycerol monolaurate, 5-10% w/w of rosemary oil, 5-10% w/w of nisin peptide, 5-20% w/w of calcium carbonate, 2-5% w/w of piperin, 2-5% w/w of sodium alginate, and 2-5% w/w of carboxymethyl cellulose for the prevention and treatment of white spot disease in shrimps.

In a yet another preferred embodiment, provided herein is a novel aquaculture feed-additive composition comprising 71% w/w of glycerol monolaurate, 10% w/w of rosemary oil, 5% w/w of nisin peptide, 5% w/w of calcium carbonate, 5% w/w of piperin, 2% w/w of sodium alginate, and 2% w/w of carboxymethyl cellulose for the prevention and treatment of diseases of white spot disease in shrimps.

In another preferred embodiment, provided herein is a novel aquaculture feed-additive composition comprising 5-20% w/w of glycerol monolaurate, 5-10% w/w of rosemary oil, 5-10% w/w of nisin peptide, 5-20% w/w of calcium carbonate, 2-5% w/w of piperin, 2-5% w/w of sodium alginate, and 2-5% w/w of carboxymethyl cellulose for the prevention and treatment of white spot disease in shrimps.

According to another embodiment of the present disclosure, provided herein is a novel aquaculture feed-additive composition comprising glycerol monolaurate, rosemary oil, nisin peptide, calcium carbonate, piperin, sodium alginate and carboxymethyl cellulose for the prevention of white spot syndrome virus infection by administering a dosage of 2-7 g/kg as a prophylactic dosage.

According to yet another preferred embodiment of the present invention, provided herein is a novel aquaculture feed-additive composition comprising glycerol monolaurate, rosemary oil, nisin peptide, calcium carbonate, piperin, sodium alginate and carboxymethyl cellulose for the prevention of white spot syndrome virus infection by administering a dosage of 5 g/kg as a prophylactic dosage.

According to another embodiment of the present invention, provided herein is a novel aquaculture feed-additive composition comprising glycerol monolaurate, rosemary oil, nisin peptide, calcium carbonate, piperin, sodium alginate and carboxymethyl cellulose for the treatment of white spot syndrome virus infection by administering a dosage of 7-15 g/kg as a therapeutic dosage.

According to a preferred embodiment of the present disclosure, provided herein is a novel aquaculture feed-additive composition comprising glycerol monolaurate, rosemary oil, nisin peptide, calcium carbonate, piperin, sodium alginate and carboxymethyl cellulose for the treatment of white spot syndrome virus infection by administering a dosage of 10 g/kg as a therapeutic dosage.

In a yet another embodiment of the present invention, provided herein is a novel aquaculture feed-additive composition comprising glycerol monolaurate, rosemary oil, nisin peptide, calcium carbonate, piperin, sodium alginate and carboxymethyl cellulose, optionally containing hyperimmunized egg powder for the prevention and treatment of diseases which include but are not limited to white spot disease in shrimps.

Yet another embodiment of the present disclosure, provided herein is an aquaculture feed-additive composition comprises glycerol monolaurate and saponin-rich extract. The saponin-rich extract of the said aquaculture feed-additive composition is derived from Rosmarinus officinalis L.

### 3. PREPARATION OF A FEED-ADDITIVE COMPOSITION

In an embodiment of the present invention, provided herein is a process for the preparation of the novel aquaculture feed-additive composition as described herein.

### 4. PREPARATION OF FUNCTIONAL FEED

Further, each embodiment described in this disclosure is provided merely as an example or illustration of the present invention and should not necessarily be construed as preferred or advantageous over other embodiments. The detailed description includes specific details for the purpose of providing a thorough understanding of the present invention. However, it will be apparent to those skilled in the art that the present invention may be practiced without these specific details. The embodiments provided herein are for the purpose of filing the present complete specification; however, further embodiments within this disclosure are possible and shall/may be covered in the complete specification.

### 4. EXAMPLES

The feed additive composition capable of acting as a prophylaxis and therapeutic composition against white spot syndrome virus (WSSV) will be described herein below in the following examples. However, the following examples are merely for illustrative purposes and has not been intended to limit the scope of the present invention.

### Example 1: Preparation of the Novel Feed-Additive Composition

The raw materials were weighed according to the following mass formulae for the preparation of feed additive.
Glycerol monolaurate (w/w): 71%
Rosemary oil (w/w): 10%
Nisin Peptide (w/w): 5%
Calcium carbonate (w/w): 5%
Piperin (w/w): 5%
Sodium alginate (w/w): 2%
Carboxymethyl cellulose. (w/w): 2%

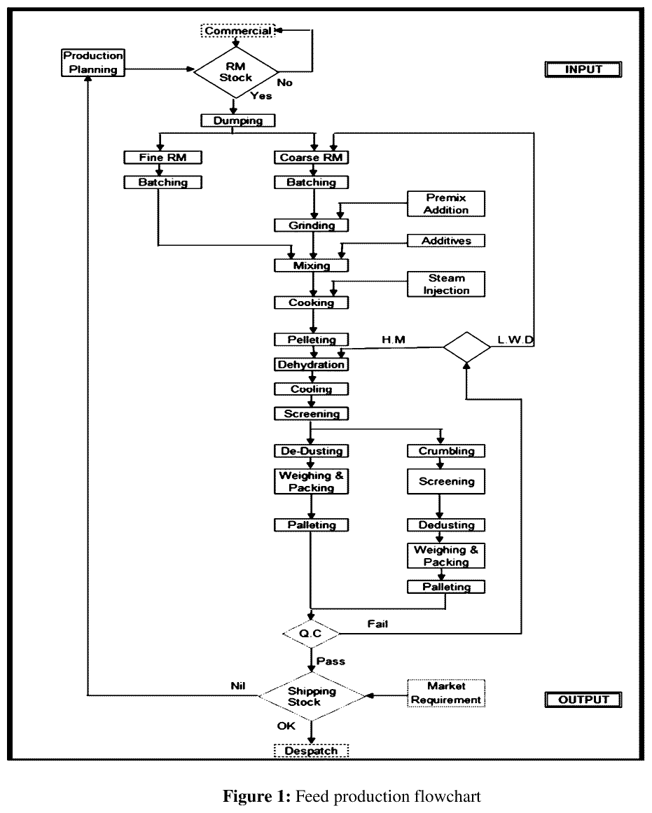

The above listed components were accurately weighed and added into the blender for geometrical mixing at 40 to 60 RPM for 30 minutes. The powder was unloaded from the blender and sieved with 60 mesh sieve and stored into air tight HDPE containers at room temperature. The process of production of the novel fee-additive composition has been illustrated in FIG. 1.

### Example 2

### 2.1 Preparation of Functional Feed, Using the Invention Disclosed in Example 1.

The feed raw materials were weighed according to the following mass formulae
Glycerol monolaurate (w/w): 5-20%,
Rosemary oil (w/w): 5-10%
Nisin Peptide (w/w): 5-10%
Calcium carbonate (w/w): 5-20%
Piperin (w/w): 2-5%
Sodium alginate (w/w): 2-5%
Carboxymethyl cellulose. (w/w): 3-5%
Fish meal (58% protein): 20-30%
High protein soy: 30-45%
Wheat floor: 20-30%
Broken rice: 5-10%
Krill meal: 5-10%
Squid meal: 5-10%
Fish oil: 5-5%
Soya Lecithin liquid: 2-5%
Imulsifier: 1-2%
Methionine: 1-3%
Lysin: 2-4%
Vitamins: 24%
Vitamin-C: 1-3%
Vitamin-E: 1-4%
Bioplex: 0.5-1%
Actigen: 0.5-1%
Potassium Chloride: 0.5-1%
Magnesium suphate: 1-3%
Salt: 2-5%
Toxin binder: 1-2%
Binder: 2-5%

The above mentioned raw materials were pulverized in a pulverizer and sieved with a 40 mesh sieve. Further, water is added and mixed with a blender according to the below mentioned detailed production flow chart with specifications. The final pellet is of 1-2 mm diameter sized.

TABLE 1
Feed production Parameters

| Production Parameters | Temp in °C | Time in Min |
|---|---|---|
| Grinding (Horizontal Pulveriser) | 45-50 | 35-45 |
| Blending (DRM/SRM) | | 10 |
| DDC - Preconditioner | 90 | 4-5 |
| Pellet mill input | 90-105 | 5 |
| Post conditioner | 100-105 | 45-60 |
| Dehydration | 85-90 | 10 |
| Entry cooler | 60 | 35-40 |
| Exit Cooler | 35 | |

The production methodology of the novel feed-additive composition has been illustrated in the form of a flow chart containing the production steps and specifications represented as FIG. 1.

### 2.2 Trial Facility and Design:

0.4 hectare earthen ponds were used for conducting this trial. Deep bore well was the source of saline water of 20 to 24 ppt. The experimental set-up was as indicated in Table 2.

TABLE 2
Experimental set-up for conducting the trial.

| Treatment Code | T1 |
|---|---|
| Pond Number | 1 |
| Water Spread area (Ha) | 0.4 |
| Stocking density (Nos./m2) | 40 |
| Number of shrimps stocked (in | |
| Lakhs) | 1, 60, 000 |
| PL stage (stocking period) | 10 |

### 2.3 Test Animals

Litopenaeus vannamei PL-10 (Whiteleg shrimp post larva) was procured from Coastal Aquaculture Authority (CAA)-approved hatchery. These shrimps were cultivated and used as test animals to assess the capability of the feed composition in treating WSD.

### 2.4 Dosage of Feed Additive

10 g of the feed composition disclosed in Example 1 was mixed with 20 mL of clean water and 20 mL of binder (Brand name: Baylife, manufactured by Water Base Ltd) and dissolved thoroughly. After complete dissolution, the mixed formulation was spared uniformly on 1 kg feed and dried for 30 minutes under shade before the feeding was done. Feeding was done four times in a day and every meal was included with 10 g of disclosed innovation per kg of feed.

### 2.5 Observation of Water Quality Parameters:

The ponds were regularly checked for water quality parameters such as on daily in house and weekly in outside lab. The water samples were collected every day at 6 am and 3 pm for analysis throughout the experimental period. Microbiological analysis, such as enumeration of Total Bacterial Count (TBC count), Vibrio—Yellow colonies and Vibrio—Green colonies were tested weekly once throughout the experimental period.

### 2.6 Pond Observation

Pond performance and observation were recorded and reported regularly, including the recommendation and use of additives, chemical supplements, etc.

### 2.7 Shrimp Health Observation:

Shrimps were examined every week during sampling and results before and after the application were noted.

### 2.8 RT-PCR Test and Periodic Sample Evaluation:

During sampling weak or dead shrimps of about 10 numbers were randomly collected for pathogen analysis (WSSV) screening by reverse transcription-polymerase chain reaction (RT-PCR). The frequency by which the sample was collected was once in seven days.

### 2.9 Evaluation of Growth Parameters:

Shrimp weight was measured during weekly sampling. The samples were collected from all the four corners of the pond using cast net and the shrimp weight was recorded. The shrimp weight of each netting was measured for Initial weight and final weight to evaluate the shrimp growth using the definition:

Weight gain (WG)=Mean final weight (g)−Mean initial weight (g)

### Specific Growth Rate (SGR):

$$SGR = \frac{( LnW 2 - LnW 1 )}{T 2 - T 1} \times 100$$

In which,
W2: Mean weight at the end of the experiment
W1: Mean weight at the beginning of the experiment
T2-T1: Duration of the experiment (days)

### Feed Conversion Rate (FCR):

$$FCR = \frac{Total feed consumed}{Total final shrimp weight - Total initial weight}$$

### Survival Rate (SR):

$$SR = \frac{N}{N 0} \times 100$$

In which:
N0=number of shrimps at the beginning of the experiment
N=number of shrimps at the end of the experiment

### Average Daily Growth (ADG)

Average daily growth (g)=body weight gain (g)/number of days

### 2.10 Mortality Rate:

Mortality in each pond was counted and recorded daily.

### 2.11 Harvest Data:

Harvest is carried out during morning at 7 AM. Harvest is planned considering various environmental parameters, such as water temperature, sunlight, salinity etc. as sudden changes will induce moulting of the shrimp. The harvested shrimps were then transported to the processing plant after recording the crate details.

### Example 3

### Preparation of Novel Feed-Additive Two-Component Composition:

### 3.1 Dose Preparation:

Negative control: The term is used herein in the meaning well-known in the art and as used herein does not have disease expression and is administered with only a buffer

Positive Control: The term is used herein in reference to disease positive control where only WSSV virus is injected without any antagonist. This positive for the disease expression.

Placebo: The term is used herein in reference to disease negative wherein the highest dose of test molecule has been injected, and evidences for safety of test molecules.

The experimental groups have been divided in seven groups, Group-I-VII as described below:

| Group | Description |
|---|---|
| I | 100 μL NTE buffer (negative control) |
| II | 96 μL NTE buffer + 4 μL of WSSV (positive control) |
| III | 86 μL NTE buffer + 4 μL viral inoculum + 10 μL of |
| | saponins rich extract at 1 mg/animal |
| IV | 86 μL NTE buffer + 4 μL viral inoculum + 10 μL of |
| | saponins rich extract at 3 mg/animal |
| V | 86 μL NTE buffer + 4 μL viral inoculum + 10 μL of GML |
| | at 1 mg/animal |
| VI | 86 μL NTE buffer + 4 μL viral inoculum + 10 μL of GML |
| | at 3 mg/animal |
| VII | 86 μL NTE buffer + 4 μL viral inoculum + 10 μL of |
| | mixture containing 3 mg of GML and 2 mg of saponins |
| | extract/animal. |

### 3.2 Materials and Methods

### 3.2.1 Animal Collection

Freshwater crabs P. hydrodromous were collected from Padavedu (12.6310070 79.052885°) Vellore, India.

### 3.1.2 Animal Preparation

The animals with a body weight of 20-25 g were transported to the laboratory and maintained at room temperature (25° C.). The crabs were fed twice a day with boiled egg whites. The crabs were kept in the described condition for one week before the experiment.

### 3.1.3 Virus Propagation

As described in earlier reports (Natarajan et al., 2017), haemolymph was drawn from WSSV infected shrimp using a 26-gauge syringe through heart puncture. The haemolymph was centrifuged at 3000 rpm for 20 minutes at 4° C. The supernatant was collected and it was centrifuged for 30 minutes at 4° C. and then it was filtered using 0.45p m and stored at −20° C. for further studies. The haemolymph were analyzed via PCR to confirm the presence of WSSV. To amplify the structural VP28 gene, a conventional PCR reaction was set up, which includes μl of 2× master mix, 5 pmol WSSV-VP28 primer (Table 1), 3 μl PCR-grade water, and 1 μl template DNA (Sundaram et al., 2016; Yoganandhan et al., 2003). The viral titer quantified through real time PCR, was 1.6×106 copies μL−1 which served as the stock for this study.

### Example 4. ANALYSIS AND OBSERVATION

### 4.1 Seed Procurement and Trial Data:

L. vannamei PL-10 prcured from a CAA-approved hatchery were tested for microscopic examination (outside lab), Real Time-PCR, salinity test, temperature test and survival test before stocking in the pond (0.4 Ha Pond No: 1 and 2) and stocking was initiated after PLs resulting the overall total score of >80% survivability.

### 3.1. Disease Outbreaks

In Pond.02 on DOC-35, moulting and 1^{st} white faecal strings were reported in the pond and decrease in the feeding ratio was also observed.

On DOC-36 1 dead piece was found floating in the corner of the pond.
On DOC-37, 6 dead pieces were found floating on the surface and no moulting was observed.
On DOC-38, mortality rate increased from 6 pieces to 13 pieces throughout the day. Further, the PCR report showed WSSV positive with 28-CT.

In connection with the positive reports, the formulation disclosed in Example 1 was applied at a dosage of 10 g/kg of feed in every meal from DOC-39. On DOC-42, moulting started, so Mg+ and K+ minerals of 20 kg and 10 kg were applied, respectively. Next day (DOC-43), there were no mortality pieces found. On DOC-44, the PCR report revealed WSSV positive with 35-CT, but no mortality was observed. Live pieces were screened for WSSV in RT-PCR on DOC-49 and results indicated negative for WSSV.

### 3.3. Water Quality Parameters:

The experimental ponds were regularly checked for all the water quality parameters, on a daily and weekly basis. The water samples were collected every day at 6 am and 3 pm for analysis throughout the experiment. All the health and pond care additives that impact the shrimp and pond performance were recorded and reported regularly, including the use of additives, chemical supplements, etc. The water quality parameters of the ponds were found to be similar as the source of water and all the environmental parameters are almost similar in the ponds that are represented in the Table 3. It was observed from this culture that there was no deterioration in the water quality after the application of formulation disclosed in example 1 along with feed.

TABLE 3
Pond water quality parameters

Pond No. 1

| Parameters | UOM | Mean | SD |
|---|---|---|---|
| Salinity | ppt | 21.0 | 1.9 |
| Dissolved oxygen | ppm | 6.0 | 0.5 |
| pH | | 7.8 | 0.3 |
| Water | °C | 30.4 | 1.0 |
| temperature | | | |
| Alkalinity | ppm | 166.1 | 15.3 |
| Ammonia | ppm | 0.1 | 0.1 |
| Transparency | cm | 46.9 | 8.5 |
| Water depth | cm | 130.0 | 4.4 |
| Yellow colonies | cfu/ml | 108.6 | 97.2 |
| Green colonies | cfu/ml | 13.3 | 5.8 |

### 3.4. Pond Observations

The types of application provided according to the pond observations have been enlisted in the Table 4 herein below. During the days of moulting, the ponds were applied with Baymin, Mg⁺ and K⁺ minerals. During the WSSV disease outbreak, the pond was treated with the disclosed novel formulation with a treatment dosage of 10 g/kg feed or prophylactic dosage of 5 g/kg feed according to the CT values reported.

TABLE 4
Pond observations and recommendations

| | White feces | | | Recommendation & |
|---|---|---|---|---|
| DOC | strings | Moulting | Mortality | Application |
| 35 | Yes | Yes | — | — |
| 36 | Yes | Yes | Yes (1) | — |
| 37 | Yes | Yes | Yes (6) | — |
| 38 | Yes | — | Yes (13) | — |
| 39 | Yes | — | Yes (15) | Formulation Example-1 at |
| | | | | 10 g/kg |
| 40 | Yes | — | Yes (52) | Formulation Example-1 at |
| | | | | 10 g/kg |
| 41 | No | — | Yes (22) | Formulation Example-1 at |
| | | | | 10 g/kg |
| 42 | No | Yes | Yes (10) | Formulation Example-1 at |
| | | | | 10 g/kg |
| 43 | No | Yes | No | Formulation Example-1 at |
| | | | | 10 g/kg |
| 44 | No | Yes | No | Formulation Example-1 at |
| | | | | 10 g/kg |
| 45 | No | — | No | Formulation Example-1 at |
| | | | | 10 g/kg |
| 46 | No | — | No | Formulation Example-1 at |
| | | | | 10 g/kg |
| 47 | No | — | No | Formulation Example-1 at |
| | | | | 10 g/kg |
| 48 | No | — | No | Formulation Example-1 at |
| | | | | 10 g/kg |
| 49 | No | — | No | Formulation Example-1 at |
| | | | | 10 g/kg |

### 3.5. Shrimp Health Observation:

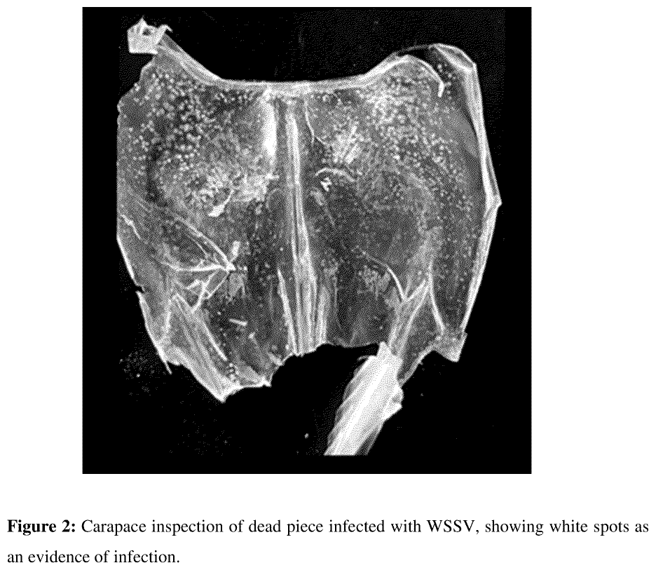

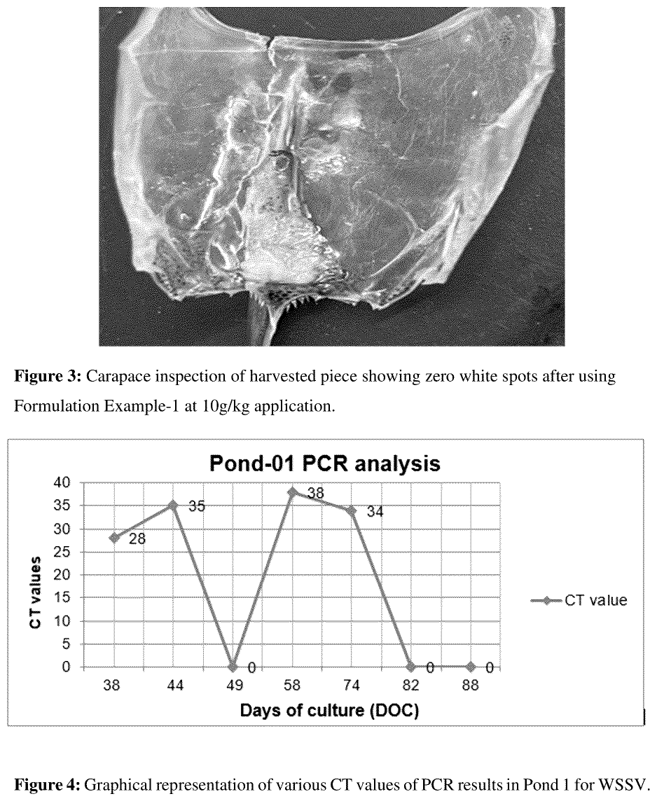

The clinical morphological appearance of the carapace of both WSSV-infected shrimp and the healthy harvested shrimp images were depicted in the FIGS. 2 and 3. The figures indicate that the prominent morphological sign of WSSV infection of having white spots were found to retard after the application of the pond with the novel composition disclosed in example-1 at the dosage of 10 g/kg.

Photographic representation of the carpases of the dead shrimps infected with WSSV and the harvested treatment group has been provided in FIG. 1 and FIG. 2.

### 3.6. Detection of WSSV in RT-PCR:

The RT-PCR technique as mentioned herein was used to detect the WSSV infection in the cultured shrimps. The below Table 5 provides the test results for the RT-PCR of the diseased shrimps. A graphical representation of the CT values of the PCR results of the treatment group in Pond 1 has been provided in FIG. 4.

TABLE 5
The RT-PCR test score at different time points against WSSV
Table 6: PCR analysis results of shrimps

Pond 1

| DOC | WSSV | CT value |
|---|---|---|
| 38 | Positive | 28 |
| 44 | Positive | 35 |
| 49 | Negative | 0 |
| 58 | Positive | 38 |
| 74 | Positive | 34 |
| 82 | Negative | 0 |
| 88 | Negative | 0 |

### 3.7. Growth Parameters:

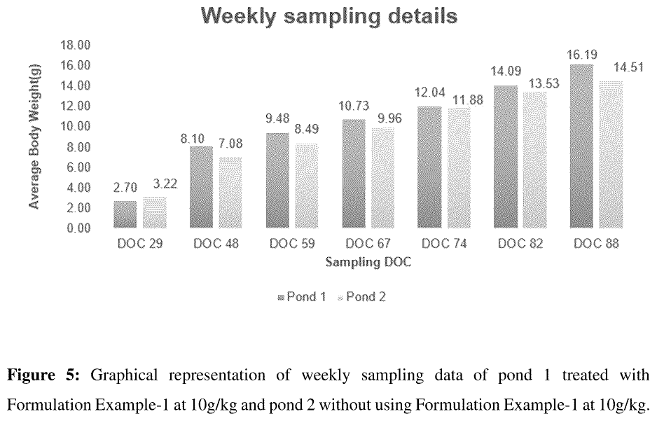

The experimental pond observations were observed and recorded during sampling days. Shrimp weight along with species count, biomass, number, ABW, ADG, SGR and survival was measured during the weekly sampling routine. The Average Daily Growth (ADG) number of L. vannamei shrimps is one of the indicators of the success of a farming process, i.e., higher the ADG number, bigger is the daily growth in gram/day unit. The ADG number is the same across all treatments (0.19-0.17 g/day). A graphical representation of the weekly sampling data of pond 1 treated with the novel composition at a dosage of 10 g/kg and pond 2 being the negative control group has been provided in FIG. 5.

### 3.8. Mortality Assessment:

Mortality of the cultured shrimps in each pond was recorded every day. The dead shrimps were removed and counted to avoid contamination of water.

TABLE 6
Mortality observations in experimental
ponds at different time points:

| DOC | Pond 1 Mortality (Pcs.) |
|---|---|
| 36 | Yes (1) |
| 37 | Yes (6) |
| 38 | Yes (13) |
| 39 | Yes (15) |
| 40 | Yes (52) |
| 41 | Yes (22) |
| 42 | Yes (10) |
| 50 | No |
| 51 | No |
| 52 | No |

### 3.9. Harvest Data:

The ponds were harvested after 92 days of culture from the date of stocking. Harvesting was carried out in the morning hours and the harvested shrimps were transported to the processing plant after evaluation.

The harvested details have been represented in the Table 7.

TABLE 7
Harvesting details of experimental pond 1
Harvesting Data

| S. No | Details | POND 1 |
|---|---|---|
| 1 | DOC | 93 |
| 2 | Stocking density / sq. m | 40 |
| 3 | Pond Size | 4000 |
| 4 | Stocking density | 1, 60, 000 |
| 5 | Average body weight(g) | 16.19 |
| 6 | Total feed (kg) | 1628 |
| 7 | Survival (%) | 75 |
| 8 | Total biomass (Kg) | 1949.9 |
| 10 | Count/kg | 65 |
| 11 | FCR | 1.42 |

### In Vivo Anti-Viral Bioassay:

The anti-viral efficacy of GMVL and saponin-rich extract against WSSV was assessed in the freshwater crab, previously acclimated to laboratory conditions. The crabs were divided into different experimental groups containing 4 crabs per group and the experiment was performed in duplicates. The viral inoculum mixture was injected into the respective group after 3 h of pre-incubation. All the crabs were injected intramuscularly at the second abdominal segment. Briefly, healthy crabs received the injection as given above. The crabs were fed twice a day and observed regularly for gross signs of disease. The experiment was carried out up to several days depending on the survival after challenging with WSSV with/without above mentioned antagonist and mortality was recorded to plot a cumulative mortality graph.

### Survival Study:

Data representing treatment of crabs with saponins is provided hereinbelow:

TABLE 8

| Survival | Positive | Saponins | Saponins | |
|---|---|---|---|---|
| Time (h) | Control | (1 mg/animal) | (3 mg/animal) | Placebo |
| 0 | 0 | 0 | 0 | 0 |
| 6 | 0 | 0 | 0 | 0 |
| 12 | 0 | 0 | 1 | 0 |
| 24 | 1 | 0 | 1 | 0 |
| 48 | 1 | 1 | 0 | 0 |
| 72 | 1 | 1 | 0 | 0 |
| 96 | 1 | 1 | 1 | 0 |
| 120 | | 1 | 1 | 0 |
| 144 | | | | |

0: Represents live animal; 1: Represents Animal Mortality; Positive Control: Virus infected animals alone; Placebo: Non-infected animal injected with test sample (Saponins) at 5 mg/animal.

TABLE 9
Data representing treatment of crabs
with GML is provided hereinbelow:

| Survival | Positive | GML | GML | |
|---|---|---|---|---|
| Time (h) | Control | (1 mg/Animal) | (3 mg/Animal) | Placebo |
| 0 | 0 | 0 | 0 | 0 |
| 6 | 0 | 0 | 0 | 0 |
| 12 | 0 | 0 | 0 | 0 |
| 24 | 1 | 1 | 1 | 0 |
| 48 | 1 | 0 | 0 | 0 |
| 72 | 1 | 1 | 1 | 0 |
| 96 | 1 | 1 | 1 | 0 |
| 120 | | 1 | 1 | 0 |
| 144 | | | 1 | 0 |

0: Represents live animal; 1: Represents Animal Mortality; Positive Control: Virus infected animals alone; Placebo: Non-infected animal injected with test sample (GML) at 5 mg/animal (non-diseased)

TABLE 10
Data representing treatment of crabs with combination
of saponins and GML is provided hereinbelow:

| | | Survival with | |
|---|---|---|---|
| Survival | Positive | Combination | |
| Time (h) | Control | (Saponin + GML) | Placebo |
| 0 | 0 | 0 | 0 |
| 6 | 0 | 0 | 0 |
| 12 | 0 | 0 | 0 |
| 24 | 1 | 0 | 0 |
| 36 | 1 | 1 | 0 |
| 48 | 1 | 0 | 0 |
| 60 | 0 | 0 | 0 |
| 72 | 0 | 0 | 0 |
| 96 | 1 | 0 | 0 |
| 120 | | 0 | 0 |
| 144 | | 0 | 0 |
| 168 | | 0 | 0 |
| 192 | | 0 | 0 |
| 216 | | 0 | 0 |
| 240 | | 0 | 0 |
| 264 | | 0 | 0 |
| 288 | | 0 | 0 |
| 312 | | 0 | 0 |
| 336 | | | |

0: Represents live animal; 1: Represents Animal Mortality; Positive Control: Virus infected animals alone; Placebo: Non-infected animal injected with test sample (Saponins + GML) at 5 mg/animal (both)

### 4. RESULTS AND CONCLUSION

After applying the novel composition disclosed in Example 1 at the dosage of 10 g/kg to pond-1 from the DOC (Days of Culture) of 38, the mortality completely ceased at DOC-49, allowing the crop to continue until the end. Further, no deterioration in water quality following the application of feed coated with formulation disclosed in example-1 was observed. Following the use of the novel formulation, the shrimp activity improved, no clinical indications of WSSV were observed, and no abnormalities were recorded as indicated in FIG. 3.

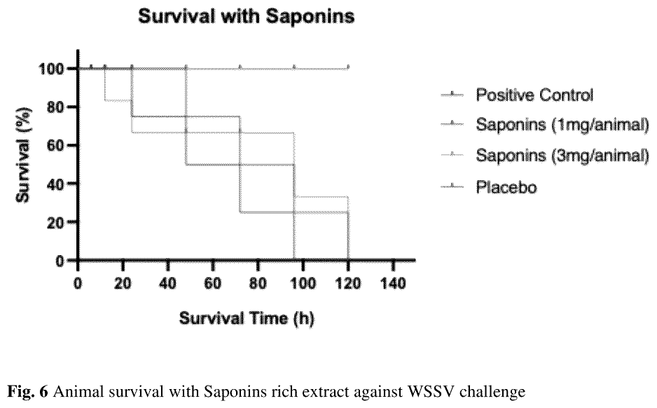

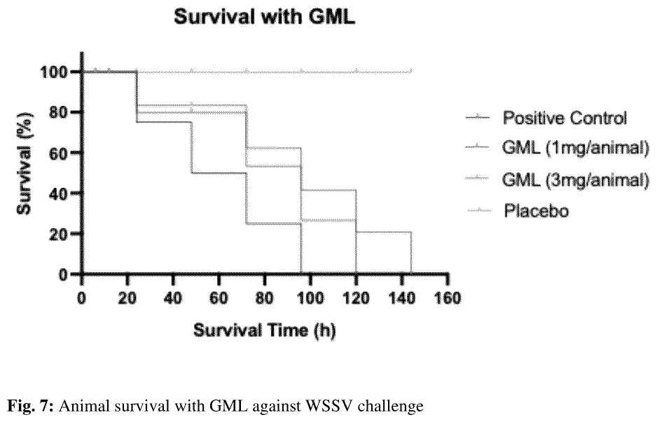

Animal survival assessment was also done against WSSV challenge with Saponins rich extract as disclosed in Example 3. WSSV virus and saponins rich extract at different concentrations were pre-incubated followed by co-injected (i.m) to crab. Post challenging, animal survival was monitored through several hours. Placebo group has received only the buffer while positive control has received only virus without saponin-rich extract. Treatment with saponin-rich extract has resulted in partial inhibition of the virus but could not protect the animal from lethality and there by all animals in the test groups died within 120 h of post challenging while positive control group animals died within 100 h of challenging with WSSV. This result was recorded as indicated in FIG. 6.

Animal survival assessment was also done against WSSV challenge with Glycerol Monolaurate (GML) as disclosed in Example 3. WSSV virus and GML at different concentrations were preincubated followed by co-injected (i.m) to crab. Post challenging, animal survival was monitored through several hours. Placebo group has received only the buffer while positive control has received only virus without GML. GML has shown partial inhibition of the virus and extended the animal survival time >140 h 3 mg/animal dosage but could not protect the animal from lethality and there by all animals in the test groups died within 150 h of post challenging while positive control group animals died within 100 h of challenging with WSSV. This result was recorded as indicated in FIG. 7.

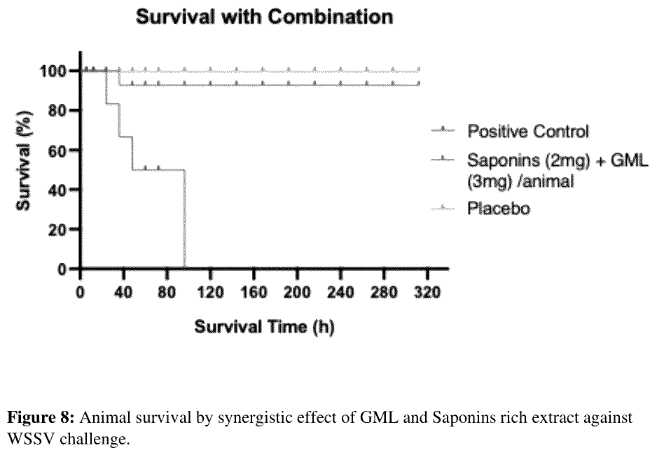

Animal survival by synergistic effect of GML and Saponins rich extract against WSSV challenge. WSSV virus was preincubated with a premixture containing Glycerol Monolaurate (3 mg/animal) and saponins rich extract (2 mg/animal) followed by co-injected (i.m) to crab. Post challenging, animal survival was monitored through several days/hours. Placebo group has received only the buffer while positive control has received only virus without any antagonists. The mixture containing both GML and saponins has shown significant inhibition on the WSSV virus and extended the animal survival time >320 h with >90% survival in the test groups, unlike the individual components tested. Positive control group animals died within 100 h of challenging with WSSV while placebo did not show any mortality in the group. This result was recorded as indicated in FIG. 8.

The interpretation of the data provided would be very clear to a person of skill in the art that the active ingredients are Glycerol Monolaurate (GML) and saponin-rich extracts, being derived from plants, such as rosemary plant. When the saponin-rich extracts and GML are treated alone, the mortality is significant. However, when the said ingredients are administered in combination, the animal survival was evidently improved and mortality is significantly reduced. The results show a synergy between the ingredients are Glycerol Monolaurate (GML) and saponin-rich extracts.

The use of the disclosed formulation in aquaculture animal health management can assist in preventing pathogenic infections and increase disease resistance efficacy of animal by boosting the immune system.

Although the experimental data disclosed herein includes 5 g/kg as a prophylactic dose, and 10 g/kg as a treatment dose, a person skilled in the art may deduce that mixtures having a range of 2-7 g/kg may be equally or more effective as a prophylactic dose, and mixtures having a range of 7-15 g/kg may equally or more effectively as a treatment dose.

In view of the present disclosure, which describes the present invention, all changes, modifications and, variations within the meaning and range of equivalency are considered within the scope of the invention. It is to be understood that the aspects and embodiment of the disclosure described above may be used in any combination with each other. Several of the aspects and embodiment may be combined together to form a further embodiment of the disclosure.

What is claimed is:

1. An aquaculture feed-additive composition comprising fatty acids, essential oils, bacteriocidin, and alkaloids effective in preventing and treating diseases, including bacterial, viral, parasitic and fungal infections.

2. The aquaculture feed-additive composition of claim 1, wherein the composition further comprises inorganic salt of Calcium, a salt of alginic acid, and cellulose derivatives.

3. The aquaculture feed-additive composition of claim 1, wherein the bacterial disease includes Vibrio species.

4. The aquaculture feed-additive composition of claim 1, wherein the fatty acids may be selected from Glyceryl Mono Laurate (GML), 1-monocaprylin (C8:0), 1-monocaprin (C10:0), 1-monolaurin (C12:0), 1-monomyristin (C14:0), 1-monopalmitin (C16:0), and 1-monostearin (C18:0) and the like.

5. The aquaculture feed-additive composition of claim 1, wherein the anti-microbial essential oil may be selected from Angelica, bergamot, lemongrass, mandarin, mint, caraway, celery, citronella, coriander, eucalyptus, geranium, petitgrain, pine, juniper, lavandin, lavander, lemon, orange, peppermint, rosemary, sage, and thyme, and the like.

6. The aquaculture feed-additive composition of claim 1, wherein the bacteriocidins may be selected from nisin, epidermin, gallidermin, Pep5, and subtilin and the like.

7. The aquaculture feed-additive composition of claim 2, wherein the inorganic salt of Calcium may be selected from calcium chloride, calcium carbonate and calcium phosphate and the like.

8. The aquaculture feed-additive composition of claim 1, wherein the naturally-derived alkaloid may be selected from piperine, capsaicin, gingerol, sinalbin, sulforaphen, isopropyl mustard oil, orxanthophylls, usnic acid, berberine, curcumin, delphinidin, hypericin, chrysarobin, esculetin, cinchonamine, cinnamaldehyde, myristic acid, limonene, amarogentin and safrole and the like.

9. The aquaculture feed-additive composition of claim 2, wherein the salt of alginic acid may be selected from sodium alginate, potassium alginate and magnesium alginate and the like.

10. The aquaculture feed-additive composition of claim 1, wherein the cellulose derivatives may be selected from cellulose esters and cellulose ethers, such as methyl cellulose (MC), hydroxypropyl cellulose (HPC), hydroxypropylmethyl cellulose (HPMC), and carboxymethyl cellulose (CMC) and the like.

11. The aquaculture feed-additive composition of claim 1, wherein the fatty acid is Glyceryl Mono Laurate (GML), the anti-microbial essential oil is rosemary oil, the bacteriocidin is nisin, the inorganic salt of Calcium is calcium carbonate, and the naturally-derived alkaloid is piperine.

12. The aquaculture feed-additive composition of claim 11, wherein the said composition comprises comprising 5-71% w/w of glycerol monolaurate, 5-10% w/w of rosemary oil, 5-10% w/w of nisin peptide, 5-20% w/w of calcium carbonate, 2-5% w/w of piperin, 2-5% w/w of sodium alginate, and 2-5% w/w of carboxymethyl cellulose.

13. The aquaculture feed-additive composition of claim 12, wherein the said concentration in w/w % is 71% of glycerol monolaurate, 10% of rosemary oil, 5% of nisin peptide, 5% of calcium carbonate, 5% of piperin, 2% of sodium alginate, and 2% of carboxymethyl cellulose.

14. An aquaculture feed-additive composition comprising glycerol monolaurate and saponin-rich extract.

15. The aquaculture feed-additive composition of claim 14, wherein the saponin-rich extract is derived from Rosmarinus officinalis L or the essential oil thereof.

16. The aquaculture feed-additive composition of claim 14, wherein the said composition comprises 5-71% w/w glycerol monolaurate and 2-20% w/w saponin-rich extract.

17. A process for production of aquaculture feed-additive composition of claim 1.

18. A process for production of aquaculture feed-additive composition as claimed in claim 14.
